(19) United States
(12) Plant Patent Application Publication
(10) Pub. No.: US 2005/0044599 P1
(43) Pub. Date: Feb. 24, 2005

(54) Variegated Illicium

(76) Inventors: Thomas H. Dodd III, Semmes, AL (US); Thayer B. Dodd, Semmes, AL (US)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/918,168

(22) Filed: Aug. 13, 2004

Related U.S. Application Data
(63) Continuation of application No. 10/156,357, filed on May 28, 2002.

(51) Int. Cl. A01H 5/00

(52) U.S. Cl. PLT/226

(57) ABSTRACT

A new and distinct form of Illicium plant, characterized by its pink flowers are variegated leaves.

### CROSS-REFERENCE TO RELATED APPLICATIONS

This application is a continuation of U.S. application Ser. No. 10/156,357, filed May 28, 2002, which is hereby incorporated herein in its entirety by reference.

### LATIN NAME OF THE GENUS AND SPECIES OF THE PLANT CLAIMED

Illicium floridanum J. Ellis forma variegatum Gaddy is a pink-flowered, variegated-leaved form of Illicium floridanum (star-anise, Florida anise).

### BACKGROUND OF THE INVENTION

The present invention relates to a new and distinct form of Illicium floridanum, referred to commercially as a Variegated Illicium.

This invention has produced a new variegated-leaved form of Illicium floridanum forma variegatum. It was propagated by shoot from Illicium floridanum forma albiflorum ‘Semmes,’ a white-flowered nonvariegated-leaved form of Illicium floridanum. Illicium floridanum J. Ellis forma variegatum Gaddy is an Illicium having variegated leaves and a light pink flower from the form of Illicium floridanum (star-anise, Florida anise).

Table 1 gives data on various taxonomic characters of the new form and typical floridanum. Characters seen in young plants of Illicium floridanum forma variegatum not mentioned in the description of typical Illicium floridanum (Vincent in Flora of North America, 1997) include asymmetrical leaves, curved leaf tips (apices), pink flower color, and leaf variegation. Differt a Illicium floridanum J. Ellis foliis variegatis et floribus roseus. (Differs from Illicium floridanum J. Ellis by its variegated leaves and by its pink flowers.). In container-grown plants less than 0.5 meter tall, leaves were significantly smaller than those of typical Illicium floridanum; however, this difference may be due to the fact that these plants are young shoots. It was propagated by shoot from Illicium floridanum ‘Alba’, variety ‘Semmes,’ a white-flowered nonvariegated-leaved form of Illicium floridanum.

This invention has not been observed under all possible environmental conditions. The phenotype may vary significantly with variations in environment such as temperature, light intensity, and day length, without, however, any variation in genotype.

### SUMMARY OF THE INVENTION

This new variety is endowed with a unique combination of traits which are determined to be the basic characteristics of this invention which in combination distinguish it as new and distinct, and materially contributes to its attractiveness. Among its important and dominant characteristics are the following: (1) the leaves of the new variety are variegated having a gray-green color at the exterior edges of the leaf and ranging from to a yellow-green to yellow at the interior of the leaf; and (2) the flowers of the new variety are light pink with the inner petals fading to a softer pink.

This invention is best compared to wild floridanum. A comparison for the leaf and flower characteristics is shown in Table 1 wherein it can be seen that the leaves both as to size and shape are distinct. Also, the flower color is distinct from the wild floridanum. The plant was 5 years old when spontaneous mutation was found. That mutation is now 12 years old.

The flower color and leaf variegation of forma variegatum has remained stable in offshoots from the original variegated-leaved plant shows that the foregoing characteristics come true to form and are established and transmitted through succeeding propagation. The plant's winter hardiness is USDA zone 6 and the drought/heat tolerance is USDA zone 10.

### BRIEF DESCRIPTION OF THE PHOTOGRAPHS

The accompanying photographs show specimens of the new variety, with their foliage and flowers in different stages of development.

FIG. 1 is a close-up photograph illustrating the variegated leaves of the new plant;

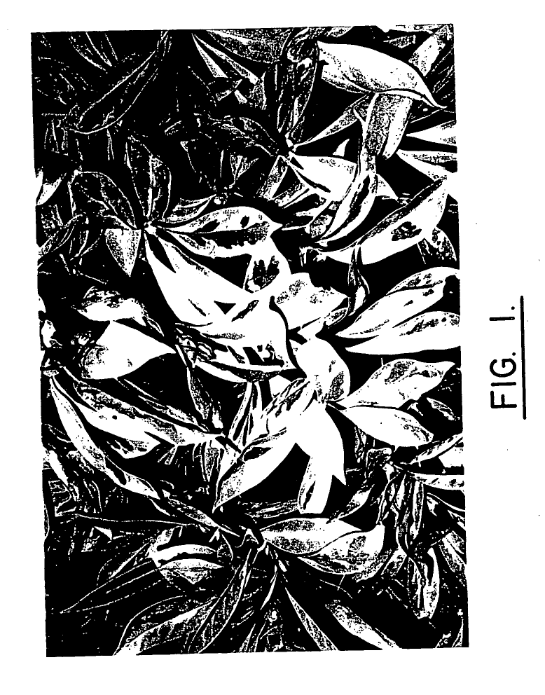

FIG. 2 is a photograph illustrating a cluster of buds of a typical plant;

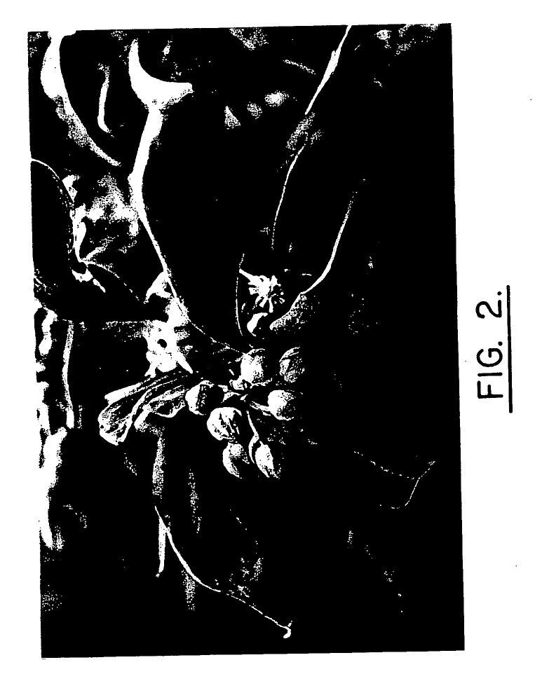

FIG. 3 is a photograph which illustrates a single flower of the new plant; and

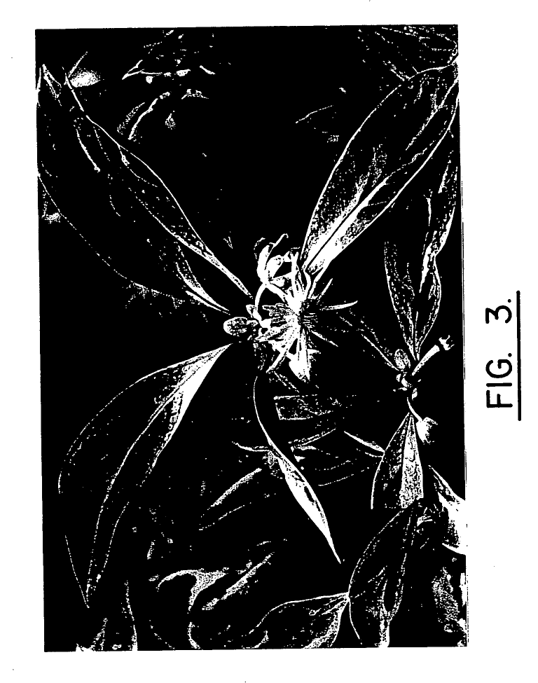

FIG. 4 is a photograph showing the plant.

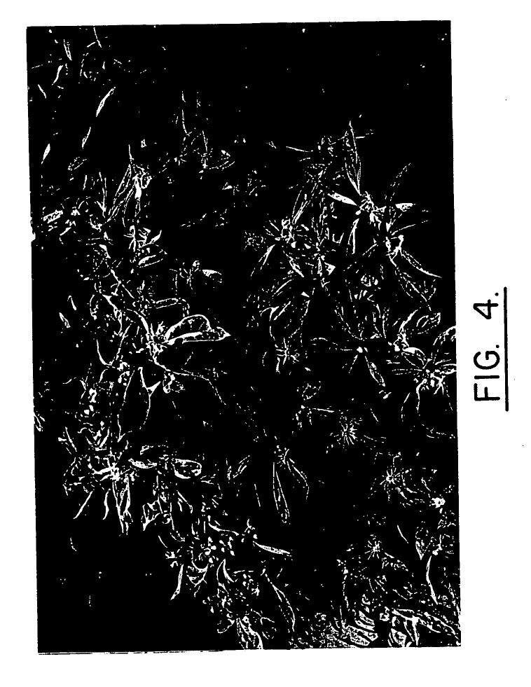

The photographs of the drawing were taken Apr. 28, 1999 in Semmes, Ala. and illustrate the typical foliage form, with color as true as possible in this type of color photograph.

### DETAILED DESCRIPTION OF THE INVENTION

The following observations, measurements and values describe plants of the new cultivar grown in Moncks Corner, S.C. in August, 1998. Color references are made in accordance with The Royal Horticultural Colour Chart of the British Color Council, except where general color terms of ordinary dictionary significance are obvious.
Origin: Dodd & Dodd Nursery Post Office Drawer 439 Semmes, Ala. 136575
   Botanical classification.—Illicium floridanum.
   Parentage.—Branch sport of Illicium floridanum ‘Alba’, variety ‘Semmes’.
   Commercial class.—Ornamental; broad-leaf evergreen.
Propagation:
   Type cuttings.—This plant has been asexually reproduced by means of semi-hardwood cuttings. All rooted cuttings have remained true to the new variety.
   Time to initiate root development.—about 4 to 5 months.
Plant description:
   Form.—Upright rounded shrub. Multi-trunked.
   Height.—6 feet to 8 feet. Slightly shorter than the average for the species.
   Trunk diameter.—original plant. — 3 inch diameter trunk.
   Tree diameter.—2 foot diameter spread.
   Bark color.—Brownish gray (RHS 199A), younger limbs dotted with varying numbers of small lenticels of color (RHS 199C); older limbs are without lenticels.
   Internodes length.—about 2 to 3 inches.
Foliage:
   Type.—Evergreen. Leaves mostly elliptic to lanceolate in shape. Apex acute to acuminate with leaf base cuneate.
   Arrangement.—Alternate. In pseudo-whorls at end of stem.
   Leaf size.—about 6.5-11 cm. long and 1.3-2.5 cm. wide.
   Leaf shape.—often asymmetrical. Entire.
   Texture.—leathery.
   Aspect.—Smooth. Aromatic anise-like odor.
   Color.—variegated with the primary green of the leaves being a grey-green — Royal Horticultural Society 189A; variegated colors range from grey-green (RHS 188B) to yellow-green (RHS 144B) to yellow (RHS 3D), often with an admixture of the latter three colors.
   Vein color.—Color RHS 190A.
   Venation pattern.—Midrib evident but lateral veins not evident.
   Abaxial surface color of the observed leaf.—Color RHS 189C.
   Leaf petiole.—the length is about 1 to 2 cm.; the width is about 0.5 cm. and the color is RHS 191D.
Flower description:
   Locality where grown.—Moncks Corner, S.C.
   Blooming habit.—Has started to flower as early as late February in Moncks Corner and continued until mid-April. Peak times have been April 1^{st}, with a tendency to repeat in summer and fall.
   Continuity.—Continuous.
   Borne.—Singly on a ½″-1½″ pedicel.
   Shape.—Round, 1″-1½″ in diameter.
   Fragrance.—Reminiscent of a wet dog or fish.
Sepels:
   Number of sepals.—6.
   Size of sepals.—1 cm. long, ¼ cm. wide.
   Color.—RHS66C.
   Flower.—20-30 strap-shaped petals, soft pink, ligulate. Pollinated by a variety of insects.
   Arrangement.—Single.
   Shape.—Round.
   Texture.—Thick, leathery.
   Aspect.—Waxy.
   Size.—1″-1½″ in diameter.
   Color.—Light pink (RHS66D) fading to softer pink (RHS69C).
   Pedicels.—the diameter is 0.2 cm. and the color is RHS 149A.
Bud:
   Size.—The bud is 1 cm. In length and ½ cm. in diameter.
   Shape.—Globular with pointed tips. Rose-pink (RHS 60D).
   Color.—Rose-pink (RHS 60D).
   Quantity of Bloom.—Abundant; outdoors.
   Peduncle.—Length about ½″-1½″.
Reproduction:
   Stamens.—Number. — 25-50.
   Pistil.—Number — 11 -21.
   Seed/Fruit.—One seeded, dehiscent follicles, 11-15 arranged in a whorl, ¾″-1″ diameter, The seed capsule is first green (RHS 144C), then yellow (RHS 150A), and finally brown (RHS 199B), maturing in November. Has a star-like configuration, distinct, seeds BB-like, brown and shiney.

Resistance to disease/susceptibility.—Resistance to disease has not been observed. The plant does not like full sun in the Southeastern U.S.

TABLE 1
Illicium floridanum forma variegatum and
its relationship to wild floridanum.¹

| | | Illicium floridanum |
|---|---|---|
| | Wild floridanum | forma variegatum |
| LEAF LENGTH | 5-21 cm | 6.5-11 cm |
| LEAF WIDTH | 1.5-6 cm | 1.3-2.5 cm |
| LEAF SHAPE | Elliptic to lanceolate | Often asymmetrical |
| LEAF BASE | Cuneate | Narrowly cuneate |
| LEAF APEX | Acute to acuminate | Often curved |
| LEAF THICKNESS | Thin to leathery | Leathery |
| LEAF COLOR | Olive | Variegated |
| FLOWER COLOR | Maroon | Pink |

¹Measurements for typical wild I. floridanum taken from Michael A. Vincent's treatment of Illicium, Pp. 59-61 In Flora of North America. 1997. Magnoliophyta: Magnoliidae and Hamemelidae. Volume 3. Oxford University Press. New York, Oxford. 590 p.

1. A new and distinct Illicium plant as herein described and illustrated.
